(19) United States
(12) Patent Application Publication
(10) Pub. No.: US 2002/0173833 A1
(43) Pub. Date: Nov. 21, 2002

(54) Apparatus and method for high energy photodynamic therapy of acne vulgaris, seborrhea and other skin disorders

(76) Inventors: Avner Korman, Herzelia (IL); Yoram Harth, Haifa (IL)

Correspondence Address: DANIEL J SWIRSKY, PO BOX 2345, BEIT SHEMESH, 99544, IL

(21) Appl. No.: 10/098,592

(22) Filed: Mar. 18, 2002

Related U.S. Application Data
(63) Continuation-in-part of application No. 10/007,702, filed on Dec. 10, 2001.
Application No. 10/007,702 is a continuation-in-part of application No. 09/756,130, filed on Jan. 9, 2001.
Application No. 09/756,130 is a continuation-in-part of application No. PCT/IL99/00374, filed on Jul. 7, 1999.

(51) Int. Cl. A61N 5/06

(52) U.S. Cl. 607/88; 607/94

(57) ABSTRACT

An apparatus and method for the phototherapy of different skin conditions, particularly acne vulgaris seborrhea and inflammation. The invention consists of a multiple session treatment method by a violet/blue light source with a spectral emission in the range of 400-450 nanometer and possible additional spectral bands in the green and red part of the spectrum and possibly with the topical application of oxygen transporting compounds, and/or a methylene blue solution. The apparatus includes at least one narrow spectral band light source with spectral emittance concentrated in the violet/blue spectral band. The apparatus use the radiation to reduce the level of extra cellular pro inflammatory cytokines related to inflammation and to significantly reduce the acne bacteria population by a photodynamic effect.

### CROSS REFERENCE TO RELATED APPLICATIONS

This application is a continuation in part application of U.S. application Ser. No: 10/007,702, filed Dec. 10, 2001, which is a continuation in part application of U.S. application Ser. No: 09/756130, filed Jan.9, 2001, which is a continuation in part application of PCT Application No: PCT/IL99/00374, filed Jul. 7, 1999.

### FIELD AND BACKGROUND OF THE INVENTION

The present invention relates to an apparatus and method for the photodynamic treatment of acne vulgaris and seborrhea and, more particularly, to a violet/blue light radiating system that illuminates a collimated narrow bandwidth beam on the treated skin area. The method relates to the combined photodynamic skin treatment including narrow band violet/blue light radiation and topical application of oxidative and/or keratolytic agents.

The present invention also relates to the phototherapy of inflammation and particularly to the phototherapy of inflammation using narrow band high intensity light source in the spectral range of 400-450 nm.

The enlargement and obstruction of sebaceous glands cause acne vulgaris. Due to the accumulation of sebum in the glands, bacteria, mainly propionibacterium acnes (p. acnes), proliferate in the glands. These bacteria cause inflammation and later the formation of pustular lesions and acne cysts, which heal by scarring.

It is known that p. acnes produce porphyrins. It is also known that visible light in the violet/blue (405-410 nanometer range), or less efficiently, red (630-670 nanometer range) are able to induce a photodynamic effect in which the porphyrins in the enlarged sebaceous glands react with oxygen to form peroxides. These peroxides are short-lived toxic compounds that are able to eliminate, or considerably diminish, the number of bacteria in the glands.

Photodynamic therapy (PDT) is based on the optimal interaction of four elements; light, photosensitizer, oxygen and skin penetration. Prior patents and publications related to acne phototherapy dealt only with the first two elements of PDT, i.e., and light exposure and sebaceous gland porphyrins. Studies have shown that the photodestruction of p. acnes is increased exponentially in an oxygen rich environment.

Various attempts have been made to treat acne with light; Mendes et al. (U.S. Pat. No. 5,549,660) described a method for the light therapy of acne using low intensity red light. Their apparatus was meant to treat acne through it effect on macrophages in the skin. Its low light intensity is not sufficient for an efficient photodynamic destruction of p. acnes in the deeper layers of the skin. High intensity visible light phototherapy for acne was described by Meffert et al., (Dermatol-Monatsschr. 1990; 176(10): 597-603) but they used a light source emitting not only visible light but also UVA comprising up to 15-20% of the total irradiation dose. Sigurdsson et al (Dermatology 1997; 94:256-260), used Philips HPM-10 400W combined with an UVILEX 390-filter (Desag. Germany) that filters most but not all ultraviolet A (UVA) harmful rays. The spectrum of their lamp peaked at 420 nanometer and had 2 other small peak of emission at 405 and 435 nanometer. Their apparatus emitted at 40 cm; 0.5J/cm² of UVA, 20 Jcm²/of violet/blue and 5 J/cm² of green None of the prior art devices aimed at the photo treatment of acne free of harmful UV radiation are able to essentially eliminate the acne lesions within a short period of four weeks in the vast majority of patients. All photo based treatments necessitates at least 3-4 month of treatment procedures resulting in increasing risks of scars. Low power photo treatment of inflammations is practiced for over 2 decades. The spectral range used for these treatments is in the red (630 nm) or the Infrared range of the spectrum. Other lamps or light sources used for the treatment of inflammations emit blue light but in conjunction with ultraviolet light. The use and intensity of these sources is limited by the erythemogenic, photoaging and photo carcinogenic effects of UV light.

High intensity, UV free, violet/blue light sources are used for the therapy of Acne, whereby p. acnes bacteria are destroyed through a photodynamic process. (International patent publication PCT number WO 00/02491).

### SUMMARY OF THE INVENTION

Basic science research has shown in vitro that the viability of p. acnes relates inversely to light intensity and to oxygen levels to which the p. acnes are exposed. Sigurdsson et al achieved with their apparatus 30% reduction of the total severity of acne and particularly 49% reduction of the number of pustules. The rate of success can be drastically improved by adding and penetrating oxygen to the skin daily and/ or immediately before skin exposure to high intensity violet/blue light.

According to the present invention there is provided an apparatus and a method for acne phototherapy, achieved by the use of a specially designed apparatus having a narrow spectral band of violet/blue light emission radiation with the radiation characterized by a high energy threshold level, above pre-determined radiation flux. In another embodiment said apparatus having at least one additional spectral line. In another embodiment the illumination of the light source flux, In the entire UV spectrum, is less than a predetermined UV safety energy threshold level.

In another embodiment the illumination flux of the light source in the UV spectrum is less than 0.1 microwatt/cm² in the spectral range of 200-400 nm.

In another embodiment the treatment with the invention apparatus can be combined with a pre-treatment application on the treated skin area of an oxygen transporting compound, based on the use of one or more of the materials from the group of compounds consisting of perfluorocarbons, oxidative substances, keratolytic substances and external photosensitizer, such as Methylene blue 0.1-5%.

The present invention apparatus utilizes in another embodiment the least one spectral band for the treatment of inflammation. This spectral band is a high intensity, UV free, narrowband violet/blue light energy in the spectral band range of 400-450 nm.

The invention apparatus adaptation for the treatment of inflammation is based on the research discovery that the violet-blue light at the same spectral bandwidth as the light applicable for the treatment of acne is also capable of reducing pro-inflammatory cytokines produced by T cells, thereby diminishing local inflammation.

It was further found that by illuminating inflamed tissues, including the case of inflamed acne lesions, with over 10 mw/cm² of light within the bandwidth of 400-450 nm, the inflammation is significantly diminished. That inflammation reduction discovery paves the way to the efficient, safe and fast therapy of large areas as well as lesional targeted areas of inflamed tissues without the risks of UV irradiation.

There is thus provided, in accordance with an embodiment of the present invention, apparatus for treatment of a skin disorder. The apparatus includes at least one light source with spectral emittance concentrated in at least one specific narrow spectral band, wherein one spectral band is in the range of 400 to 450 nm, an optical system for collecting and shaping light emitted from the at least one light source and an electronic unit to control parameters associated with the spectral emittance from the at least one light source.

Furthermore, in accordance with an embodiment of the present invention, the parameters include at least one of a group including duration, power and emitted spectral bands of the light source emittance.

Furthermore, in accordance with an embodiment of the present invention, the apparatus further includes a mechanical fixture for holding the light source at an adjustable distance and direction related to a treatment area.

Furthermore, in accordance with an embodiment of the present invention, the illumination energy of the light source flux, is higher than a predetermined threshold level. The threshold level is a level required for biological destruction of acne and seborrhea causing factors, Specifically this treatment energy threshold level enables the destruction of the P. acne bacteria population to a much lower level, so as to get a significantly reduced post treatment bacteria population size.

The invention apparatus illumination energy flux level triggers a photodynamic treatment process that creates the condition in which the acne bacteria destruction rate surpasses the natural bacteria population average multiplication and growth rate, thus ensuring that the balance of the bacteria population will be significantly further reduced from one treatment to another, until total bacteria population reduction ratio will provide treatment results wherein post-treatment bacteria population will be at least four (4) orders of magnitudes lower than the pre-treatment bacteria population size.

Furthermore, in accordance with an embodiment of the present invention the invention apparatus for the treatment of acne is capable of reducing over 60% of the original number of acne lesions in average within four weeks, without radiating any UV illumination on the treated skin area and without causing any damage to this skin.

Furthermore, in accordance with an embodiment of the present invention, the illumination energy threshold level of the illumination light source is at least 40 mw/cm² at a distance from the light source of 30 cm.

Furthermore, in accordance with an embodiment of the present invention, the illuminated area on a patient body includes an illumination area large enough to illuminate an infected typical size skin area from a fixed position of the light source related to the skin area. In accordance with an embodiment of the present invention The illuminated area is at least 400 cm².

Additionally, in accordance with an embodiment of the present invention, the apparatus further includes an illumination head having at least two converging collimated beams from at least two directions, each of the beams generated by a separate light source positioned at a distance from the other at least one light source.

Additionally, in accordance with an embodiment of the present invention, the apparatus further includes a computer controlled imaging unit for imaging an illuminated treated area and for monitoring by counting lesions on the treated area, using computerized counting techniques.

Additionally, in accordance with an embodiment of the present invention, the apparatus further includes a computer controlled display unit for displaying the imaged illumination treated area, wherein counting is carried out by an operator marking lesions on the display of the illumination treated imaged surface area. Alternatively, the computer lesions counting by image processing techniques to detect and count each lesion in the illumination treated imaged surface area. The score of the computer lesion counting is recorded in a computer memory to enable monitoring the lesion healing process through a series of consecutive treatments.

Furthermore, in accordance with an embodiment of the present invention, the computer controlled imaging unit display image includes at least one of a group includes a graph of the number of counted lesions versus accumulated treatment time and a table consisting of number of counted lesions in each treatment session.

Furthermore, in accordance with an embodiment of the present invention, the apparatus further includes at least one optical element of a group includes a liquid filled light guide, a solid transparent light guide, a fiber bundle light guide and an array of lenses and mirrors for collecting and conducting the light source radiation and illuminating the skin treated area at an adjustable distance, energy density and direction.

Furthermore, in accordance with an embodiment of the present invention, the light source is a Gallium, Mercury and halides gas mixture discharge lamp with peak emission in the 405-440 spectral band. Alternatively, the light source is an Excimer lamp with peak emission in the spectral range of 400-430 nm.

Alternatively, the light source is composed of one ore more of a group of light sources including a metal halide gas discharge lamp, an excimer lamp, an Ion Krypton gas laser with a spectral emission in the range 405 to 440 nm, a diode and a diode matrix. The diode or diodes are selected from the group consisting of violet/blue laser diodes, and light emitting diodes (LED) with narrow spectral band emission in the spectral range of 405-440 nm, or 520-570 nm, or 625-670 nm, or any combination thereof.

Furthermore, in accordance with an embodiment of the present invention, the light is collected and projected by at least one reflector, wherein the reflector is selected from the group includes of an elliptical cross-section cylindrical reflector, parabolic cross-section cylindrical reflector, and an asymmetric aspheric reflector.

Alternatively, the light is collected and further collimated by a set of two orthogonal cylindrical lenses.

Furthermore, in accordance with an embodiment of the present invention, the light of the at least one light source is collected by an elliptical cross-section reflector having a first focal point and a second focal point. The light source is disposed at the first focal point and has disposed at the second focal point a slit shape aperture of a slit to circular beam shaping and conducting light guide.

Additionally, according to the present invention there is also provided a method of treating a skin disorder. The method includes providing a light radiation source having spectral characteristics of at least one of a group of narrow spectral bands consisting of violet/blue (405-440 nm), red (630-670 nm) and green (520-550 nm) light, applying a compound to a skin area, illuminating the skin area with the light radiation source, and additionally illuminating the skin area after a predetermined time period.

Furthermore, in accordance with an embodiment of the present invention, the skin disorder is one of a group including acne and seborrhea.

Furthermore, in accordance with an embodiment of the present invention, the compound is selected from a group consisting of a topical oxygen transporting perfluoroocarbon, an oxidative agent, a keratolytic agent and a methylene blue solution

Furthermore, in accordance with an embodiment of the present invention, the predetermined time period is at least 24 hours.

Furthermore, in accordance with an embodiment of the present invention, the method further includes a pretreating application of the compound, concentrating the light on the skin area by an optical system and a mechanical fixture, and exposing the skin area at specific time intervals.

Furthermore, in accordance with an embodiment of the present invention, the time interval is 1-5 weekly exposure to violet/blue light for typically 2-10 weeks, with a minimum 24 hour's time gap between exposures.

Furthermore, in accordance with an embodiment of the present invention, the step of illuminating is accomplished by projecting on the skin area with an illumination power in the range of 10 mW/cm² to 50 mW/cm² of violet/blue light radiation.

Furthermore, in accordance with an embodiment of the present invention, the compound is hydrogen peroxide in the concentration of 1-10% by weight and the concentration of salicylic acid is 1-10% by weight.

Furthermore, in accordance with an embodiment of the present invention, pretreating is carried out daily or alternatively immediately before light exposure.

Furthermore, in accordance with an embodiment of the present invention, the material is selected from the group consisting of oxidative and keratolytic compounds is in an aqueous gel. Alternatively, the material selected from the group consisting of oxidative and keratolytic compounds is in oil in water emulsion.

Furthermore, in accordance with an embodiment of the present invention, the oxidative and/or keratolytic compound is within a material selected from the group consisting of a liposome and a positively charged submicron emulsion. Alternatively, the oxidative and/or keratolytic compounds is in a Propylene glycol 10-50% base or an oil in water emulsion mixed with molecular oxygen that is sprayed continuously on the skin before or during light exposure.

Furthermore, in accordance with an embodiment of the present invention, methylene blue 0.1-5% in distilled water or gel bases is applied to the skin before or during light exposure.

It provides a way to increase the photodestruction of p. acnes by providing and illuminating the affected area with high intensity monochromatic, or multi-spectral discrete emission lines light energy, exactly matching the optimal action spectrum of the photosensitizer created by the p. acnes.

Methylene blue is a dye used parentally for treatment of methemoglobinemia in newborns and topically for disinfecting of skin. In vitro and in vivo studies have shown that Methylene blue may be activated by light to induce a photodynamic reaction. Methylene blue was used for the inactivation of herpes virus helicoabacter pillory and for the experimental therapy of skin bladder and esophageal cancers. The method of photodynamic therapy may also be enhanced by adding an external photosensitizing agent such as Methylene blue in a concentration of 0.1-5%.

The proposed method significantly increases the oxygen pressure in the sebaceous glands through the use of oxygen transporting compounds based on perfluorocarbons and/or oxidative emulsions. The proposed method also enhances light and compound penetration into the skin using translucent gels and keratolytic agents. The proposed apparatus emits light energy above a biologic bacteria destruction threshold. The light source generates a high intensity non-coherent light in the exact narrow spectral band or bands, needed for the activation of the photodynamic reaction while flittering out the harmful UV light. This narrow and specific wavelength range radiation enables the administration of sufficient intensity of light to the deeper layers of the dermis without excessive heat formation in the epidermis. The required spectral band is emitted by the present invention light source for the photodynamic destruction of p. acnes in the acne sebaceous glands.

In another embodiment of the invention, the apparatus is used for the reduction of inflammation, wherein the output power spectral band is further broadened to the 400-450 nm spectral range.

In yet another embodiment of the invention, apparatus, the light source is capable of treating any skin disorder related to inflammation from the group including; soft tissue inflammation, muscular inflammation, post traumatic soft tissue and muscular pains, arthralgia, skin ulcers such as diabetic ulcers and stasis ulcers, contact dermatitis, atopic dermatitis and systemic and localized scleroderma.

In yet another embodiment of the invention, apparatus the output radiation spectrum is UV free.

In yet another embodiment of the invention, apparatus at least one light source is a Gallium, Mercury and halides gas mixture discharge lamp with peak emission in the 400-450 spectral band.

In yet another embodiment of the invention, apparatus, at least one light source is selected from the group including Ion Krypton gas laser with a spectral emission in the range 400 to 450 nm, and a diode, wherein the diode is selected from the group consisting of violet/blue laser diodes, and light emitting diodes (LED) with narrow spectral band emission in the range 400-450 nm.

In another embodiment of the invention, method the skin disorder is inflammation; and (a) the light radiation source having spectral characteristics that contain at least radiation energy in the narrow band of violet/blue (400-450 nm); and (b) the light source having energy output flux over a predetermined threshold level.

In yet another embodiment of the invention, inflammation treatment method the output flux predetermined threshold level is 10 mW/cm2.

In yet another embodiment of the invention, inflammation treatment method the step of illuminating is accomplished by projecting on said skin area with an illumination power in the range of 10 mW/cm2 to 100 mW/cm2 of violet/blue light radiation.

In another embodiment of the invention, method the treated area is larger than 2 cm×2 cm.

In another embodiment of the invention, method, the light radiation source is capable of reducing level of extra cellular pro-inflammatory cytokines.

In another embodiment of the invention, method cytokines are reduced by an amount of at least 70% of the reduction achieved by UVB at the same energy radiation conditions.

In another embodiment of the invention, method the light radiation source is capable of treating skin ulcers.

In another embodiment of the invention, method the treatment is performed in conjunction with a topical formulation compound which enhances lesional tissue partial oxygen pressure.

In yet another embodiment of the invention, method the compound is selected from a group including a topical oxygen transporting perfluoroocarbon, an oxidative agent such as Hydrogen Peroxide formulation, a keratolytic agent and a Methylene blue solution.

In another embodiment of the invention, the treatment is performed in conjunction with Hyperbaric oxygen therapy.

In another embodiment of the invention, the treatment is performed in conjunction with mechanical debridment of tissue i.e. mechanical, chemical, by high pressure water jet or ultrasonic waves

In yet another embodiment of the invention, treatment requires a predetermined time period of 1-5 weekly exposures to violet/blue light for typically 1-10 weeks, with a minimum 24 hour's time gap between exposures.

In yet another embodiment of the invention, the step of illuminating is accomplished by projecting on said skin area with an illumination power in the range of 10 mW/cm2 to 100 mW/cm2 of violet/blue light radiation.

### BRIEF DESCRIPTION OF THE DRAWINGS

The invention is herein described, by way of example only, with reference to the accompanying drawings, wherein:

FIG. 1 is a schematic front and side view illustrations of one embodiment of the photodynamic treatment apparatus according to the present invention.

FIGS. 2A and 2B are schematic side view and front view illustrations respectively of the illumination head unit, the same embodiment of the present invention apparatus wherein the illumination unit head structure is based on a violet/blue light source of a gas discharge lamp;

FIGS. 3A and 3B are schematic top and bottom views respectively of the light source unit in the apparatus of FIG. 1, in an embodiment wherein the illumination unit structure is based on a circular array of LED's, or laser diodes, emitting a narrow spectral band red light illumination component, the array is integrated on the perimeter of a parabolic cross-section reflector, in the focal point of which is situated a high illumination intensity, narrow spectral band, violet/blue light gas discharge light source;

FIG. 4 is a schematic bottom view illustration of the present invention violet/blue light source, in another embodiment, wherein the illumination unit structure is based on a two dimensional array of LED's, or laser diodes, emitting a preferred narrow spectral band violet/blue light illumination component, the two dimensional array can also include any spatial distribution combination of violet/blue narrow spectral band emitting laser diodes or LED's, together with red light LED's, or laser diodes emitting in the preferred red spectral band;

FIG. 5 illustrates a typical spectral distribution of the light energy emitted by the present invention dedicated violet/blue light source, in the embodiments wherein the light source is a gas discharge lamp;

FIG. 6A-6C illustrate another set of an additional three preferred embodiments of the illumination head in the apparatus according to the present invention, wherein all these embodiments are based on the application of a single axis elliptical cross-section cylindrical reflector, in the first focal point of which is fitted the illuminating gas discharge lamp arc. The image of the gas discharge light source arc is created in the second focal point of the elliptical reflector and can be then directly used for object illumination, or collected and further conducted by a fiber optic slit to circular beam shaping bundle, or collected and reshaped by a dedicated set of two orthogonal cylindrical lenses, to optimally conduct and collimate the light energy on the patient's treated skin areas;

FIG. 7 illustrates the results of the proposed apparatus, operated under laboratory controlled tests on p. acne, showing a decrease in propionibacterium acnes in 4-5 orders of magnitude, after two 30, or 60 minutes exposures separated by 72 hours of dark incubation;

FIG. 8A and 8B are two schematic views illustrations of another embodiment the present invention light source apparatus, wherein in FIG. 8A the illumination source is operated through a single illumination head;

FIG. 9A and 9B are two schematic views illustrations of another embodiment the present invention light source apparatus, wherein in FIG. 9A the illumination unit is structured of a dual illumination head configuration;

FIG. 10A and 10-B, C and D are four schematic side views illustrations of another embodiment of the present invention light source apparatus, wherein in FIG. 10A the illumination head is structured of an integrated dual illumination source;

FIG. 11B is a close look of the computerized control panel in FIG. 10A;

FIG. 11A is a back-side view of the present invention apparatus;

FIGS. 12 & 13 are graphical illustrations of the test results on the reduction In the number of lesions in the treatment of p.acnes using the apparatus of FIG. 1;

FIG. 14 demonstrates the results of the treatment by the apparatus of FIG. 1, for acne lesion reduction rate of over 60% in average, within a period of 4 weeks, versus other treatments results; and

FIG. 15 is a graphical illustration of the oxygen enhancement using different compound formulations for the treatment of p. acnes.

### DESCRIPTION OF THE PREFERRED EMBODIMENTS

The present invention is an apparatus, which can be used for photodynamic treatments in phototherapy. Specifically, the present invention can be used for the non-invasive treatment of acne vulgaris and seborrhea, thereby enabling treating various parts of the patient's body with ability to control the illumination power, energy spatial distribution, exposure duration and illumination source emittance spectral bands.

The principles and operation of the apparatus for phototherapy treatment according to the present invention may be better understood with reference to the drawings and the accompanying description.

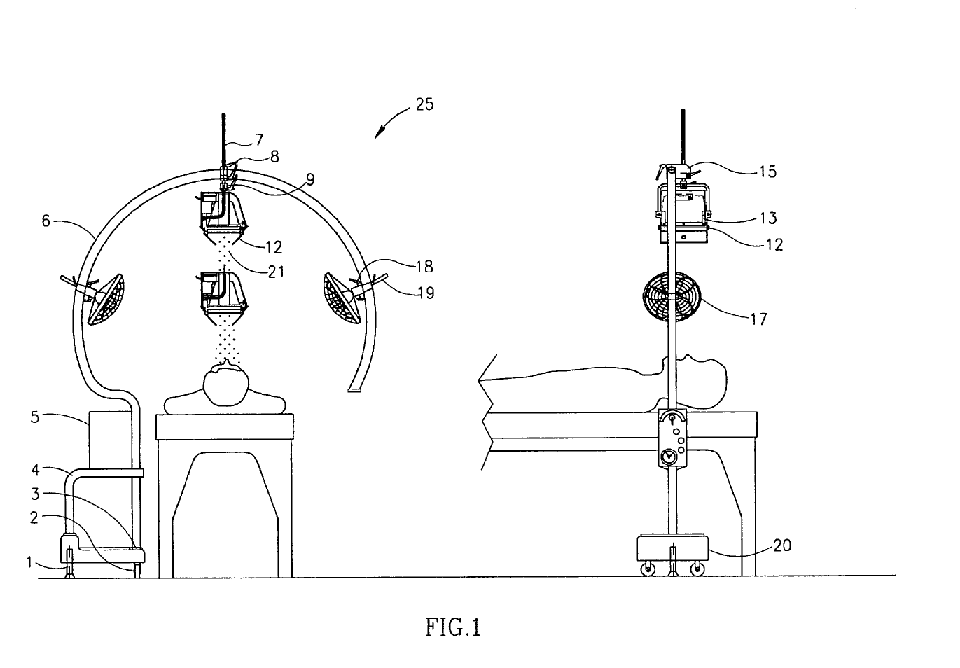

Referring now to the drawings, FIG. 1 is a schematic front and side view illustration of the photodynamic treatment apparatus according to the present invention, which is referred to herein below as system 25.

System 25 includes a violet/blue light source fixture 13, which can be moved repositioned and directed to the treated patient specific skin area by adjustment unit 15. It can also be lifted up or lowered down in order to change the effective radiated energy flux on the treated area, by using pole unit 7 and handle 8. The apparatus light source is mounted on a mechanical arc shaped fixture 6 for holding and supporting the light source at an adjustable distance and direction relative to the patient's treated skin area. The apparatus mechanical fixture 9 allows horizontal, vertical and radial placement and directing of light beam 21 from the light unit 13 to the patient's treated part of the body.

The high energy output, the consequent effective radiation flux and the relatively long distance of the treatment light source from the treated skin areas (typically in the order of 20-40 cm), is facilitated by the above described light source fixture 13. This apparatus light source fixture 13 thus enables an effective large skin area treatment in a single exposure, while typically using a fixed position for the light source fixture 13 during the entire treatment session of large skin areas, or entire human organs, as is clearly illustrated in FIG. 1.

Unit 17 is a schematic presentation of an air blower or a fan that serves to cool and remove access heat from the treated skin area. Units 18 and 19 are mechanisms to adjust the required position of unit 17.

Unit 5 is a control board for the apparatus enabling control of lamp power, illumination duration, air cooling operational parameters and general on/off and mains control functions.

Units 4 and 20 are a structural element and a balancing weight to stabilize the apparatus in a vertical up-right position. Unit 3 is a mechanical axis around which the entire apparatus arc shaped structure 6 can be rotated and refitted in any required horizontal angular position related to the treated patient bed 22.

Wheel 2 and pole 1 are elements required to move and refit the position of the apparatus according to the operational needs of the system operator.

Light source fixture 13 of consists of a lamp or a laser light source that emits violet/blue light with a peak at 405-420 nm. Close to a hundred percent of the light source ultra violet/blue light (UV) is filtered out by an integrated optical system. In a preferred embodiment of the light fixture 13, the illumination flux of the light fixture in the UV spectrum is less than 0.1 microwatt/cm² in the spectral range of 200-400 nm, to comply with UV free safety level requirements.

The required narrow spectral emission band of violet/blue light source is radiated by the present patent dedicated arc lamp due to a special gas mixture as described below, within the lamp, or by a gas laser source, or by a violet/blue light emitting semiconductor diode junction. The above light sources in a single source type embodiment, or in a combination of two or three type of light sources, allows optimal violet/blue light radiation with or without additional narrow spectral band lines in the red or green parts of the spectrum. The present invention light source enables the minimization of heat production at the treated target to a max of 23 degrees Celsius on the epidermis at 30-40 cm. A mechanical shutter 12 in front of the light source 13 may be used to exactly define the treated area.

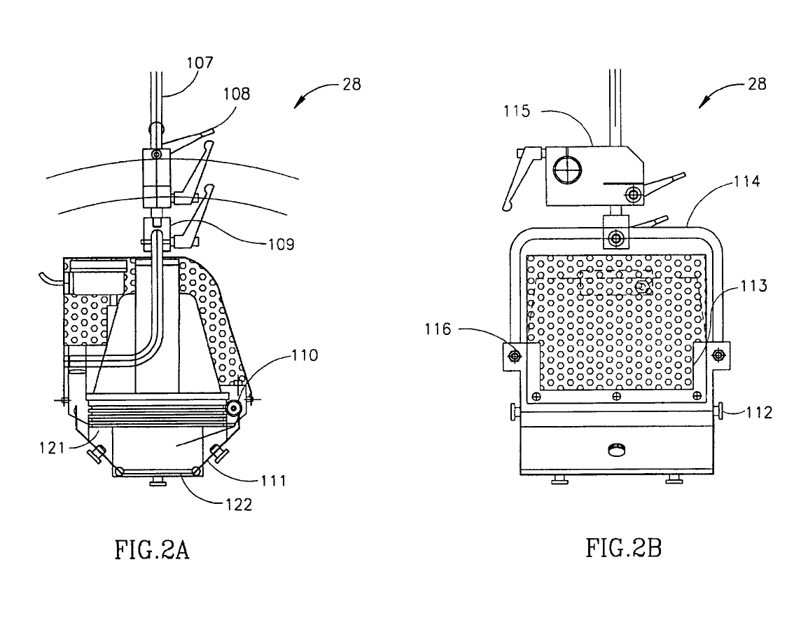

FIGS. 2A and 2B are schematic side and front view illustrations of the illumination head unit 13 according to the present invention, referred to herein below as system 28.

Illumination system 28 includes a filter unit 121 for filtering out the radiated energy spectral part, which is out of the preferred specific bandwidth in the violet/blue and/or the red spectrum, as previously described in the above background paragraph of the invention. Unit 111 is a set of four mechanical flaps with a control knob 112 and a pivoting axis 110 that create together an adjustable aperture iris unit to control the size and collimation parameters of system 28 radiated light beam. U shaped arm 114 holds and supports the illumination unit housing 113. Unit 109 enables rotation of the system 28 around vertical pivot axis 107 and to lock it in the preferred rotational angle. Unit 115 enables changing position by sliding and further fixing in a preferred position system 28 along the apparatus support arc 106. Unit 115 also enables sliding system 28 up or down and then fixing its position. Unit 122 is an optional mechanical support housing and a lens for focusing and concentrating the system 28 illumination beam on a smaller area of the treated skin, thus creating a higher light energy flux whenever required for a specific treatment.

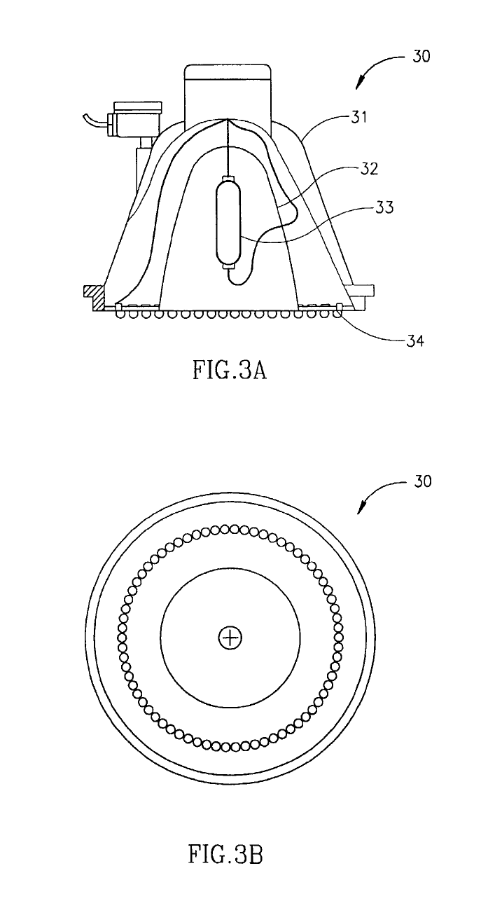

FIGS. 3A and 3B are schematic top and bottom views of another preferred embodiment of the present invention lighting head unit 13 of the apparatus described in FIG. 1, referred to herein below as system 30.

System 30 includes a housing unit 31 and a reflector 32 having preferably a parabolic vertical cross section. The gas discharge lamp, or the Excimer lamp 33 is assembled into reflector unit 32 in a way that fixes the center of the lamp illumination arc in the focal point of the reflector. Lamp 33 may be a specially designed Gallium and Lead halides gas mixture discharge lamp with peak emission in the 405-430 spectral band, or a dedicated Excimer lamp with peak emission in the spectral range of 400-430 nm.

Unit 34 is a circular array of red emission LED's or red light laser diodes installed around the aperture perimeter of the reflector unit 33.

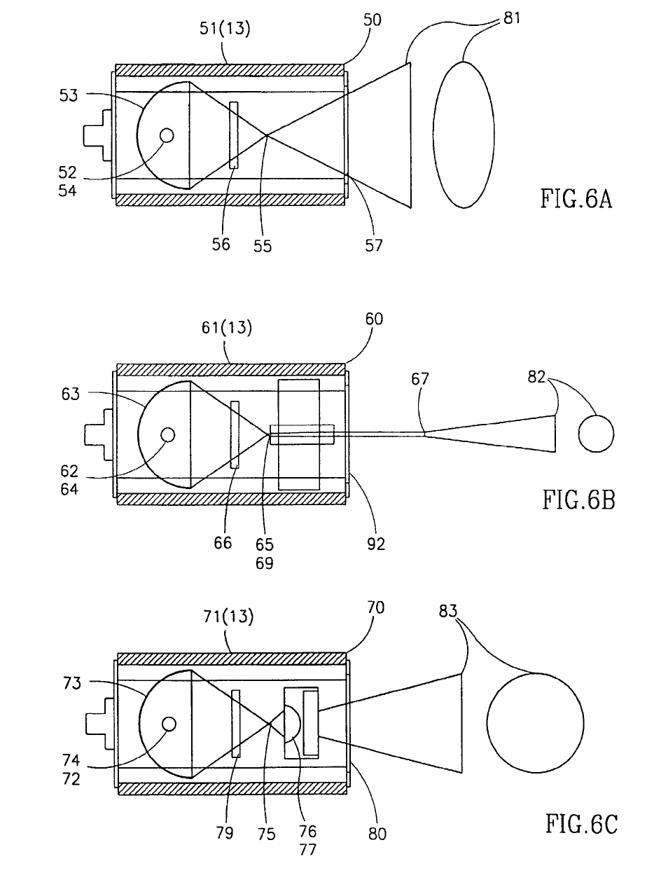

The typical effective illumination output energy of most industrial types of metal-halogen gas-discharge lamps and Excimer lamps is relatively high and is in the order of tens to hundreds of Watts. This energy level of the light radiating source, when properly collected and directed by the optical reflector 32 in this embodiment, or by other alternative types of optical light collection and direction sub-systems, such as, but not limited the configuration of the optical sub-system composed of reflector 73 and lenses 76 and 77 of FIG. 6C following, can generate an energy output flux having a typical flux value variable in the range of 10-500 mw/cm square on any spot in the illuminated skin area of the treated body. This energy flux range is high enough to facilitate an effective photo-therapeutic effect on an entire treated human limb or body part, typically over 400 cm square in area.

The Excimer lamps are produced by Heraus Nobellight from Hanau, Germany, Rhombic Corporation from the USA and Quark Ltd; they all use a highly efficient photon emission reaction (7-50%) from excimers to produce wavelengths of vacuum ultra violet (VUV) to visible light. An excimer is an excited state in a molecule that dissociates into an unbound state. This feature means that self-absorption in the lamp is small, and because of this, the lamp can be scaled to large volumes without severe degradation of the emission wavelength. The pioneering excimer lamp technology developed by Columbia Research Instruments is now owned by Rhombic Corp. It efficiently transfers the energy of electricity to microwaves and microwaves to excimers (transfer efficiency between 50 and 90%). This technology produces light of a pure wavelength more efficiently than any other light source. This is significant because light is used to induce chemical and phtobiological reactions that are wavelength specific. The excimer lamp is orders of magnitude more cost effective (dollar/photon bandwidth) than anything that current technology is capable of producing.

State of the art other UV to visible light sources include lamps like mercury, xenon, argon, tungsten halogen, fluorescent, incandescent and arc lamps and in parallel various types of lasers. As a concept these competitors are not capable of producing high efficiency narrow band excimer radiation. For all other technology based light sources the photons that are beyond the range of a chemical reaction's requirements are wasted. Rhombic was a pioneer in excimer lamps, companies such as Heraeus Noble Light, and Quark Ltd., have since come into the market.

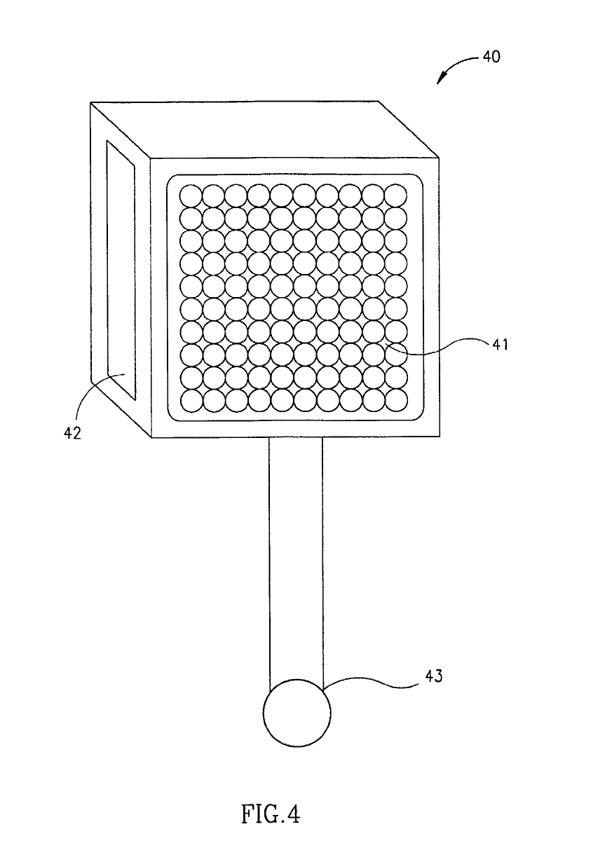

FIG. 4 is a schematic illustration of another preferred embodiment of the present invention lighting head unit 13 of the apparatus described in FIG. 1, referred to herein below as system 40.

System 40 includes a housing unit 42 and a two-dimensional array of LED's, or laser diodes 41, emitting a narrow spectral band violet/blue light illumination component. These semiconductor solid state light sources can be GaN or ZnSe components. The two-dimensional array can also include narrow spectral band red light LED's, or laser diodes, emitting in the preferred red spectral band. Unit 43 is a mechanical structure for attaching system 40 to the apparatus of FIG. 1.

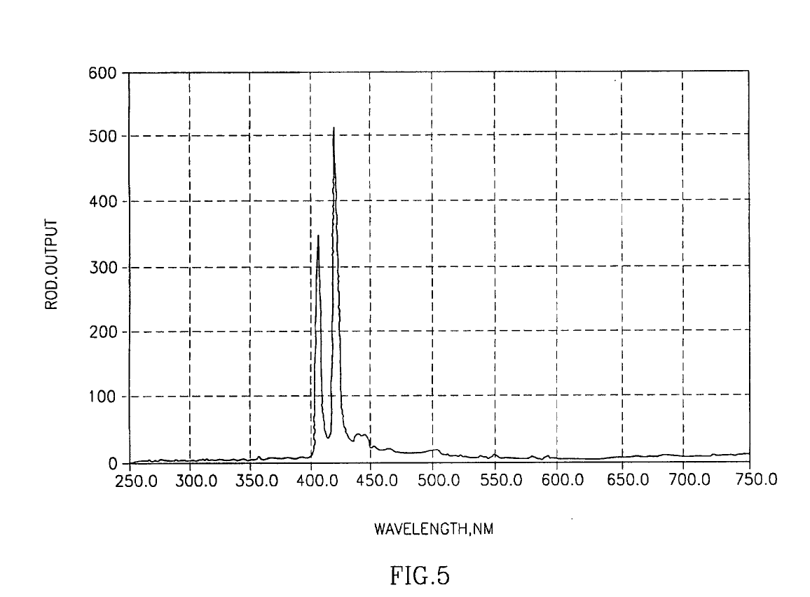

FIG. 5 illustrates a typical spectral distribution of the light energy emitted by the present invention dedicated violet/blue gas discharge lamp based light source embodiment, before further spectral optical filtration is done, in the embodiments wherein the light source is a gas discharge lamp.

FIG. 6A is a schematic cross section illustration of one of a set of three possible preferred embodiments of the present invention lighting head unit 13 of the apparatus described in FIG. 1, the first possible embodiment is referred to herein below as system 50. Light source head embodiment of system 50 consists of a housing 51 that supports an arc lamp, or an Excimer lamp, or a line beam shape laser light source 52 that emits violet/blue light with a peak at 405-420 nm. The light source is fixed in the first focal point 54 of an elliptical cross section shape reflector 53. The energy emitted out of the preferred spectral band reflected by the elliptical shaped reflector and is imaged as a line source at its second focal point 55. From the secondary focal point the beam is diverging at a small angle and creates an oval shaped illumination area 81 of typical size 20×10 cm. at a convenient treatment distance of 40 cm. from the lamp housing exit aperture. The non violet spectral part of the light source emission is rejected and filtered out by filter unit 56 and the lamp housing is sealed by tempered glass window 57 possibly coated with a heat mirror layer for the protection of the patient against heat and explosion. The required narrow spectral emission band of violet/blue light source is radiated by the present invention dedicated arc lamp due to a special gas mixture within the lamp, or by a violet/blue light emitting semiconductor diode junction array. The above light sources in a single source type embodiment, or in a combination of two or three type of different spectral emission bands light sources alternative embodiment, allows optimal violet/blue light radiation with, or without additional narrow spectral band lines in the red or green parts of the spectrum.

FIG. 6B is a schematic cross section illustration of a second possible preferred embodiment of the present invention lighting head unit 13 of the apparatus described in FIG. 1, the second possible embodiment is referred to herein below as system 60. Light source head embodiment of system 60 consists of a housing 61 that supports an arc lamp, or a line beam shape laser light source 62 that emits violet/blue light with a peak at 405-420 nm. The light source is fixed in the first focal point 64 of an elliptical cross section shape reflector 63. The energy emitted out of the preferred spectral band reflected by the elliptical shaped reflector and is imaged as a line source at its second focal point 65. In the secondary focal point the beam enters a slit shape fiber bundle aperture, matching the size and shape of the imaged light line at this point 68. At the exit circular aperture 67 of this fiber bundle the emerging light is diverging at a typical 40 degrees angle and creates a circular shaped illumination area while its size and consequently the illumination power density can be controlled by changing the distance from the exit fiber end 67 to the patient treated skin area. The non violet spectral part of the light source emission is rejected and filtered out by filter unit 66 and the lamp housing is sealed by a cover window 69. The above light sources in a single source type embodiment, or in a combination of two or three type of different spectral emission bands light sources alternative embodiment, allows optimal violet/blue light radiation with, or without additional narrow spectral band lines in the red or green parts of the spectrum.

FIG. 6C is a schematic cross section illustration of a third possible preferred embodiment of the present invention lighting head unit 13 of the apparatus described in FIG. 1, the third possible embodiment is referred to herein below as system 70. Light source head embodiment of system 70 consists of a housing 71 that supports an arc lamp, or a line beam shape laser light source 72 that emits violet/blue light with a peak at 405-420 nm. The light source is fixed in the first focal point 74 of an elliptical cross section shape reflector 73. The energy emitted out of the preferred spectral band reflected by the elliptical shaped reflector and is imaged as a line source at its second focal point 75. After passing through in the secondary focal point 75 the beam is entering a set of two cylindrical lenses 76 and 77, which are orthogonal oriented with respect to their linear axis. At the exit of this lens system aperture 78 a close to a circular light illumination area is created of typical size 20×20 cm. at a convenient treatment distance of 40 cm. from the lamp housing exit aperture. The non violet spectral part of the light source emission is rejected and filtered out by filter unit 79 and the lamp housing is sealed by a cover window 80. The above light sources in a single source type embodiment, or in a combination of two or three type of different spectral emission bands light sources alternative embodiment, allows optimal violet/blue light radiation with, or without additional narrow spectral band lines in the red or green parts of the spectrum.

The method according to the present invention improves the results by adding oxygen transporting compounds based on perfluorocarbons and/or oxidative and /or keratolytic agent, daily and or immediately pretreatment. The proposed oxygen transporting agents i.e., perfluorocarbons lipophilic emulsion, release nascent oxygen directly into the sebaceous glands achieving a 76% O₂ environment as compared to the atmospheric 20%. The proposed oxidative agents i.e., emulsion or gel of H₂O₂ 1-10%, release by contact with the enzyme cathalase present in the skin nascent oxygen. The specific formulations of the emulsion or gel prevent the upward release of the oxygen and cause a short temporary inward pressure of up to 15 Atm. of O₂, penetrating to the sebaceous glands situated in the deeper layers of the skin.

The oxygenation of the skin during the phototherapy process raises the efficiency of the desired photodestruction of p. acnes and thus decreases of acne lesion number and severity. Added keratolytic agent (i.e. 1-5% salicylic acid) to the applied formulation will enhance diffusion of O₂ into the sebaceous glands. Cooling of the applied emulsion or gel minimizes the heat in the epidermis thus allowing a further increase of the light intensity in the sebaceous glands.

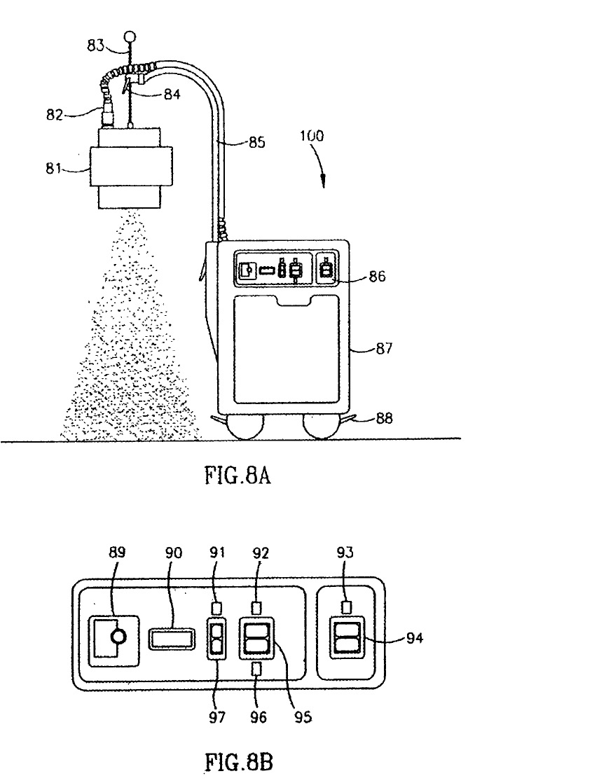

FIGS. 8A and 8B are illustrations of two schematic views of another embodiment the present invention violet/blue light source apparatus 100, wherein in FIG. 8A the apparatus 100 illumination source is installed within and operated from a single illumination head 81. The illumination head 81 is operated by a power supply and electronic control unit 87 and is supported by an adjustable height supporting mechanism 85. Lever 84 enables further fine adjustments of the lamp head 81 distance from the treated area, by sliding up or down and tightening at the requested position the support pole 83. Cable harness 82 connects the illumination head 81 to the power supply and electronic control unit 87. Control panel 86 enables the operation and control of the operational parameters of the power supply and electronic control unit 87. Unit 87 is supported by a set of four maneuvering wheels 88, having an integrated stop and fix in place mechanism.

FIG. 8B is a close look of control panel 86 in FIG. 8A. 89 is an electronic timing mechanism for controlling the treatment time. Counter 90 is a time-laps numerical indicator, for counting the accumulated operational hours of the illumination head 81. Switch 97 and indication lamp 91 control the operation of a fan cooling module, integrated in the illumination head, having the task of cooling the slightly heated illuminated skin area, during the treatment time. Switch 95 and indication lamps 92 and 96 enable switching and selecting the intensity of illumination between two discrete pre-selected energy levels. Switch 94 and the attached status indication lamp 93 is the system main power switch.

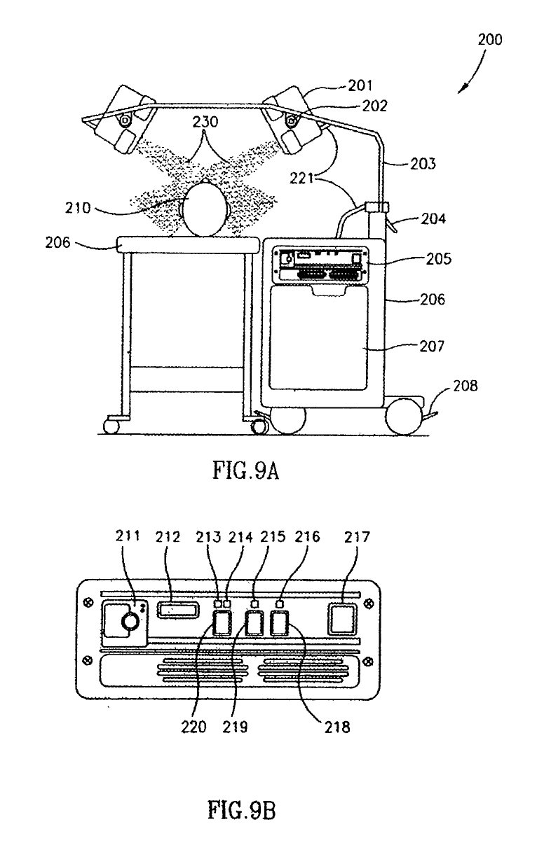

FIGS. 9A and 9B are two schematic views illustrations of another embodiment the present invention violet/blue light source apparatus 200, wherein in FIG. 9A the illumination source is structured of a dual illumination head module 201. The dual illumination head module 201 is operated by an integrated power supply and electronic control unit 206 and is supported by an adjustable height supporting mechanism 203. The head 210 height positioning related to the treated area 206 is done by sliding up or down a supporting pole with an integrated piston unit which is a part of the support mechanism 203 and then tightening the lever 204 at the requested height. Cable harness 221 connects the illumination heads 201 to the power supply and electronic control unit 206. Control panel 205 enables the operation and control of the operational parameters of the power supply and electronic control unit 206. Unit 206 is supported by a set of four maneuvering wheels 208, having an integrated stop and lock mechanism. The two illumination heads can slightly vertically tilted by the operator around pivot axis 202, in order to adjust the positioning and consequentially the illumination energy distribution of the two illumination collimated light beams 230, to be equally and evenly distributed on the two face sides of the treated patient 210.

FIG. 9B is a close look of control panel 205 in FIG. 9A. 211 is an electronic timing mechanism for controlling the treatment time. Counter 212 is a time-laps numerical indicator, for counting the accumulated operational hours of the dual beam illumination head module 201. Switch 220 and indication lamps 213 and 214 enable treatment duration control through timer 211 in one switch position, or unlimited operation time, by switching to the other switch position. Switch 219 and indication lamp 215 control the operation of a fan cooling module, integrated in the illumination head, having the task of cooling the slightly heated illuminated skin area of patient 210, during the treatment time. Switch 218 and indication lamps 216 enable switching and selecting the intensity of illumination between two discrete pre-selected energy levels. Switch 217 is the system self illuminated, main power switch.

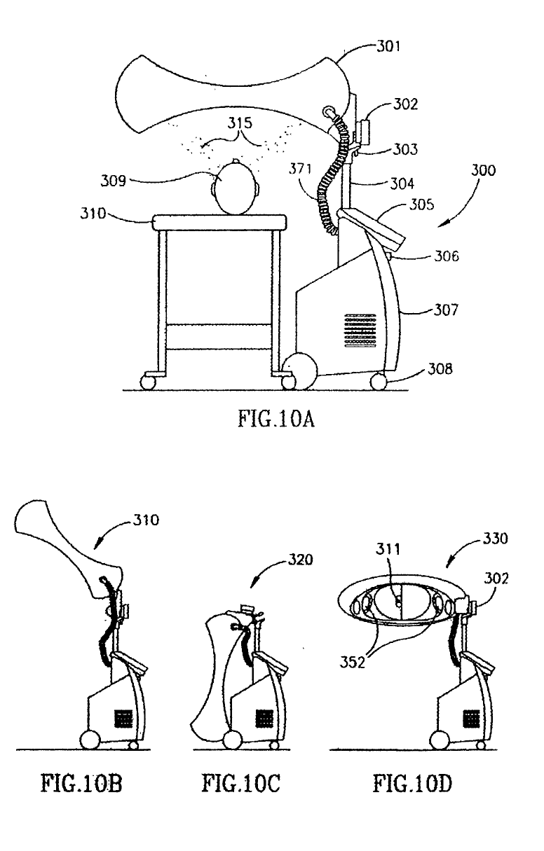

FIGS. 10A and 10-B, C and D are four schematic side views illustrations of another embodiment of the present invention violet/blue light source apparatus 300, wherein in FIG. 10A the illumination source is an integrated dual illumination source head 301. The dual illumination source head module 301 is operated by a power supply and electronic control unit 307 and is supported by an adjustable height, supporting mechanism 304. The adjustment of the lamp heads unit 301 distance from the treated area 310 and the patient treated skin area 309, is done by sliding up and down through the operation of an electro-mechanic piston to reach the requested position of the support pole 304. Cable harness 371 connects the illumination heads 301 to the power supply and electronic control unit 307. Digital video-graphic display control panel 305 enables the operation and control of all the operational parameters of the power supply, the electronic control and the computer modules of unit 307. Unit 307 is supported by a set of four maneuvering wheels 308, having an integrated stop and lock mechanism. The two illumination units inside the illumination head 301 can be slightly vertically tilted by the operator in order to adjust the positioning and consequently the illumination energy distribution of the two illumination collimated light beams 315, to be equally and evenly distributed on the two face sides of the treated patient 309.

FIG. 10B, is the second of the four schematic side views of the present invention apparatus, showing another optional functional position of the illumination head, illustrating the present invention violet/blue light apparatus embodiment of FIG. 10A.

In FIG. 10B, the illumination head folding optional position 310 enables the up-tilting of the illumination head to a position required for temporary clinic storage periods and for the patient better maneuverability after treatment session completed to support post-treatment quick patient release requirements.

FIG. 10C is the illustration of the apparatus 300 in position 320, wherein the head is folded down to minimize size and packing volume for long term storage and for packaging and transportation.

In FIG. 10D, the illumination head of the apparatus 300 is pivoted around axis 302, thus enabling the head swivel to support in any requested “roll” angle position, this feature is desired to enable treating of a patient in a seated or partially-lying position. FIG. 10D also illustrates the integrated imaging module of the apparatus 300. Imaging sequence is first done is done by a miniature digital video and sill camera 311, installed in the center of the illumination source head 301. The acquisition step of the patient treated skin area picture by camera 311, is followed by the digital image processing, analysis and related treatment progress parameters evaluation by the apparatus 300 integrated computer module.

FIG. 10E is a close look of the illumination output-window aperture 352 of the illumination head 301 and on the ventilation air input duct aperture 354, of the illumination head 301. 350 is a halogen or tungsten filament lamp, geared for the illumination of the patient treated area, illumination is required for the optimal image condition during computer controlled pre treatment imaging. 351 is the illumination unit glass protected output aperture window.

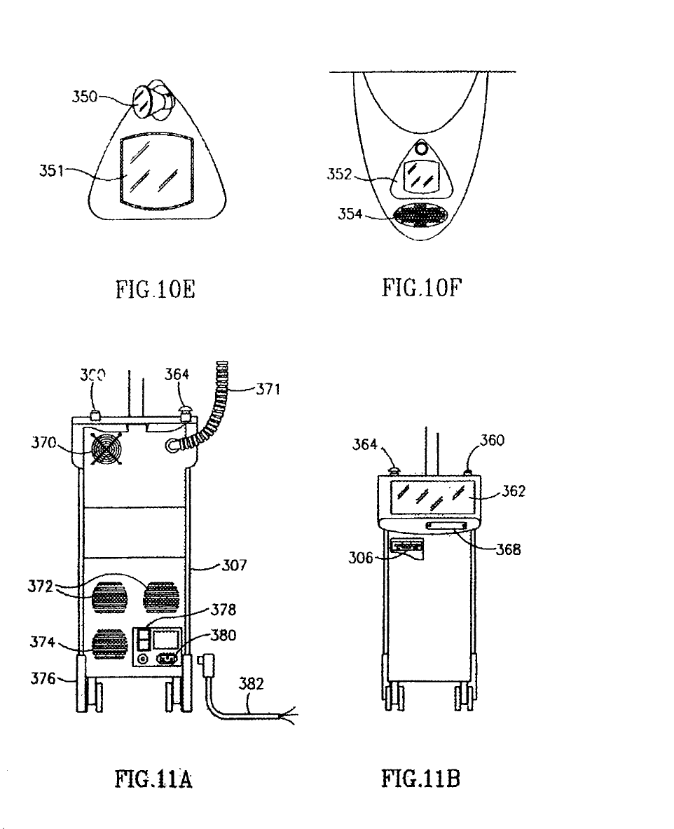

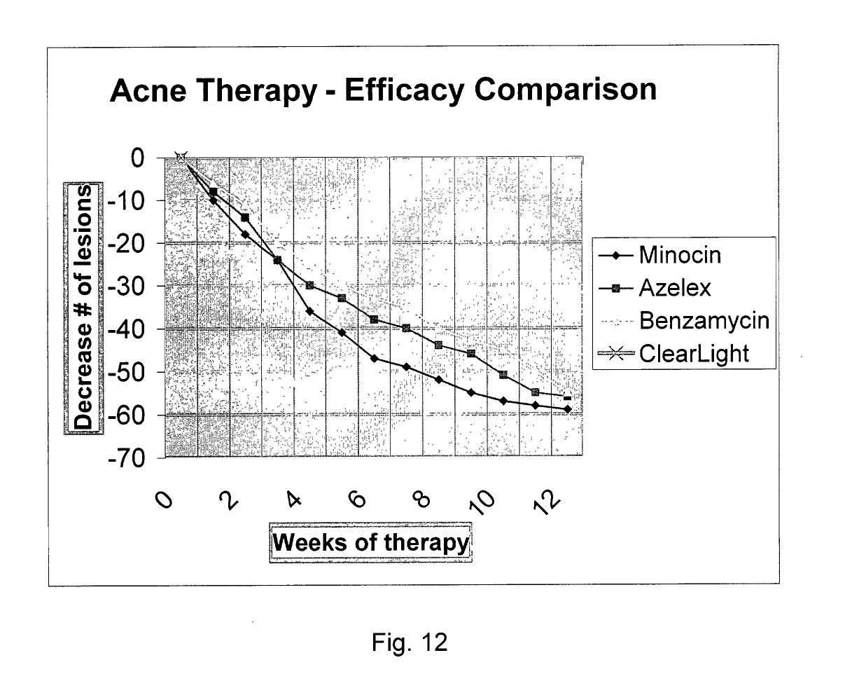

FIG. 11B is a close look of the control panel 305 in FIG. 10A. 362 is a digital video-graphic display unit having preferably an overlaid touch screen unit that supports the registration and the X-Y positioning parameters of the operators touching point in any co-ordinate location on the screen. By touching the screen 362 on discrete points, when the treated skin area still image is displayed on the screen 362, the operator can electronically mark the affected areas, or points, on the treated skin area. The computer module of unit 307 can further accumulate the number and position information of the affected points and areas and further process this information to create and display on the apparatus screen 362 any required monitoring data regarding the healing effect progress from treatment to treatment. Switch 360 is the system main on-off switch that controls the system awakening and shutting-off processes through wire operating special commands on the system computer. 364 is a Panic switch that cuts-off the input power of the mains supply to the unit, in case of emergency. The computer module of unit 307 operates an adjustable electronic timing mechanism for controlling the treatment time. 306 is a Floppy diskette drive through which treatment data is downloaded from the system and back-up or revised software versions are uploaded to the apparatus 300 computer module.

FIG. 11A is a back-side view of system 300. 370 and 374 are ventilation units for the cooling of the computer and the electronic sub-units of the control unit 307. Switch 378 is the system main power/safety on-off circuit breaker switch. 372 are two loudspeakers for generating computer synthetic voice commands and instructions to the system operator and the treated patient. Cable harness 371 is connecting and conducting the power lines and the control commands from the electronic and computer unit 307 to the illumination head 310.Cable 382 is the mains supply cable and plug and 380 is the power input socket.

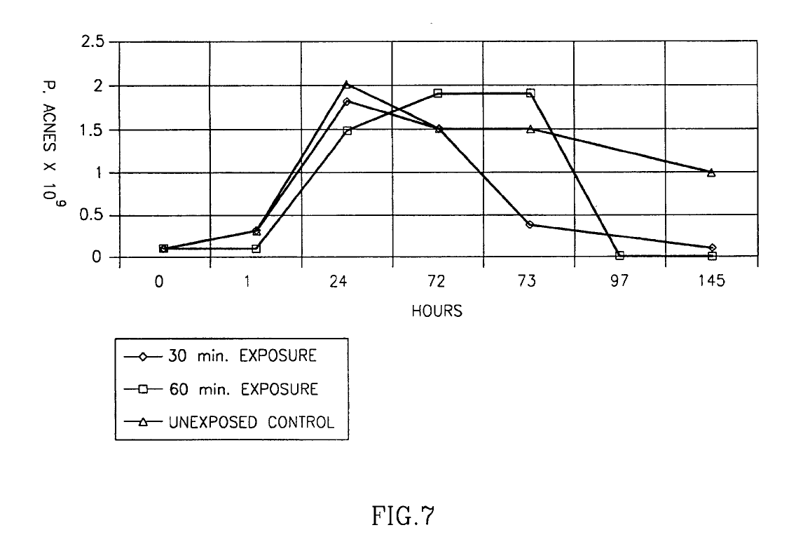

In Vitro Experimentation (Referring now to FIG. 7)

Photodestruction of p. acnes:

Bacterial strain—The strain used in this study was Propionibacterium acnes 6919 which was obtained from the American Type Culture Collection (ATCC) at Rockville, Md. U.S.A.

Growth media-Propionibacterium acnes was grown on Reinforced Clostridial Agar from Oxoid (Basingstoke, Hampshire England) at pH=6−6.2.

Illumination tests were carried out when bacteria were grown in Reinforced Clostridial Broth which was prepared from the same ingredients except the agar at pH 6−6.2.

Illumination method—Illumination was carried out by CureLight's acne therapy system. Under blue light at the wavelength of 407 nm Illumination periods were 15 minutes, 30 minutes or 60 minutes as indicated in the text. Lamp intensity was 20 mW/cm².

Bacterial growth and illumination—Propionibacterium acnes was transferred from the bacterial stock into Reinforced Clostridial Agar Plates. Bacteria were streaked on the plated for isolation of single colonies by the “clock plate technique”. These plates were called “Start plates” and were incubated for three days under aerobic conditions in an anaerobic jar. The jar contained Aaero Gen sachets from Oxoid, England to maintain anaerobic conditions suitable for P. acnes.

From the “start plates” single colonies were transferred into Reinforced Clostridial Broth and allowed to grow anaerobically for 24 hours or for 72 hours. Bacteria grown for 24 hours were defined as “young culture” and those grown for 72 hours were defined as “old culture”. The “young” or the “old” cultures were transferred to illumination for the indicated times. Some cultures were illuminated again after 24 hours or 48 hours from the first illumination as indicated in the results. After each illumination a sample was taken out from the culture and viable bacteria were counted. Viable bacteria were monitored and calculated by counting the colony forming units after appropriate dilution in saline and cultivation on the Reinforced Clostridial Agar plates under anaerobic conditions for three days. The colony forming units of the survivals were calculated per ml. Untreated cultures served as controls.

Results have shown that exposure to the proposed apparatus achieves a decrease in propionibacterium acnes from 10⁹ to <10⁴ after two 30, 60 minutes exposures separated by 72 hours of dark incubation, as shown in FIG. 7.

In addition, the destruction of p. acnes may be further enhanced by adding methylene blue 0.5% to the broth prior to irradiation.

Extensive pre-clinical tests were performed at Bar-llan University—Natural sciences lab by Prof. Zvi Malik and Prof. Yeshayahu Nitzan.

Result

illumination of “young” cultures—Propionibacterium acnes which was grown on a “start plate” was transferred into Reinforced clostridial Broth and incubated for 24 hours. After this period two illumination courses of 30′ minutes each were carried out in an interval of 48 hours from the first to the second illumination. The results demonstrate a decrease of 1 order of magnitude in viability of the culture in comparison to the control. When the culture taken from the “start plate” was grown for 24 hours and illuminated twice, this time for 60 minutes each and 48 interval between the illuminations, two orders of magnitude decrease in viability were demonstrated.

Illumination of “old cultures”—Propionibacterium acnes which was grown on a “start plate” was transferred into Reinforced Clostridial Broth and incubated for 72 hours. Bacterial cultures were illuminated once for 15 minutes or once for 60 minutes. Illumination in both illumination periods resulted in a decrease in the cultures viability of one order of magnitude. In addition, illumination of the old culture for two times for 30 minutes in an interval of 24 hours resulted in the decrease in viability of two orders of magnitude. When the “old” culture is illuminated twice for 60 minutes at an interval of 24 hours a decrease of four orders of magnitude is demonstrated in their viability.

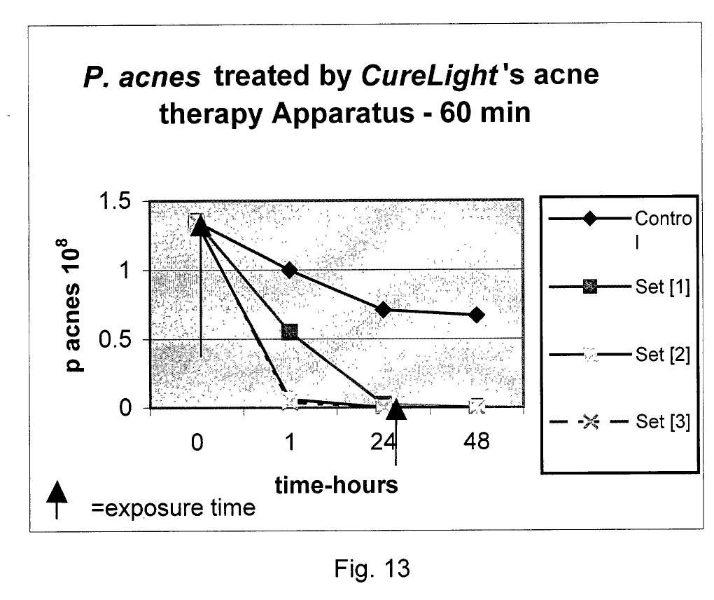

As shown in FIGS. 12 and 13, significant destruction of p. acnes was achieved up to 4 orders of magnitude.

The above laboratory experiments results prove that treatment of the acne bacteria with violet-blue light energy above a threshold energy level enables the destruction of the acne bacteria population to a much lower level, so as to get a significantly reduced post treatment bacteria population size. The invention apparatus energy flux supports bacteria destruction rates that surpasses the natural bacteria population average multiplication and growth rate between consecutive exposures, thus ensuring that the balance of the bacteria population will be significantly further reduced from one treatment to another, until total bacteria population reduction ratio will provide treatment results wherein post-treatment bacteria population will be at least 4 orders of magnitudes lower than the pre-treatment bacteria population size.

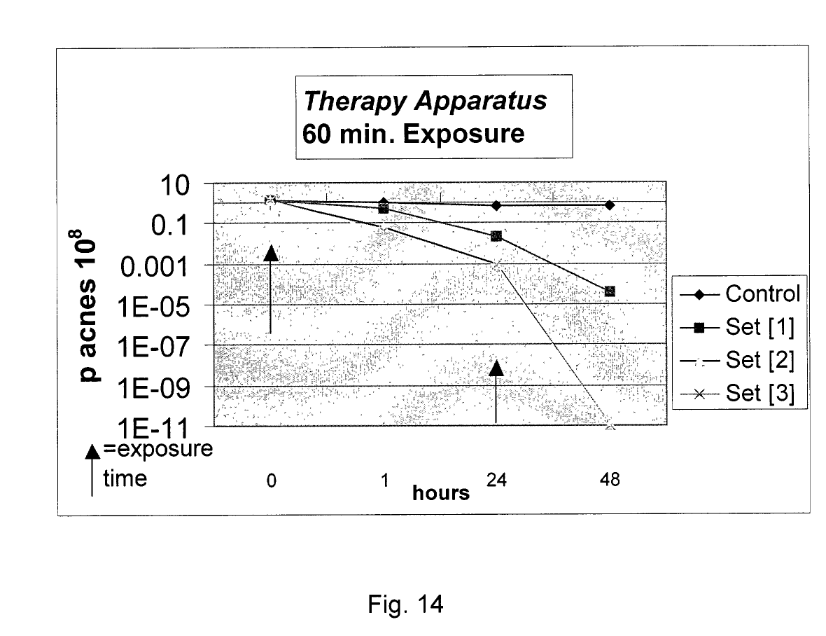

Clinical experiments have been performed on the population of over 100 patients in four clinical centers while using the invention apparatus. The results were compared to the state of the art acne lesion treatments. The results shown in FIG. 14 demonstrate an acne lesion reduction rate of over 60% in average in a period of 4 weeks, 2 treatments per week, of 15 minutes per treatment. These results should be compared to the same 60% reduction achieved in 12 weeks of daily treatments by prior art treatments.

In vivo Efficacy Testing of Hydrogen Oxygen Emulsion

Topical formulations were investigated in the Hyperbaric Chamber unit Elisha Hospital in Haifa.

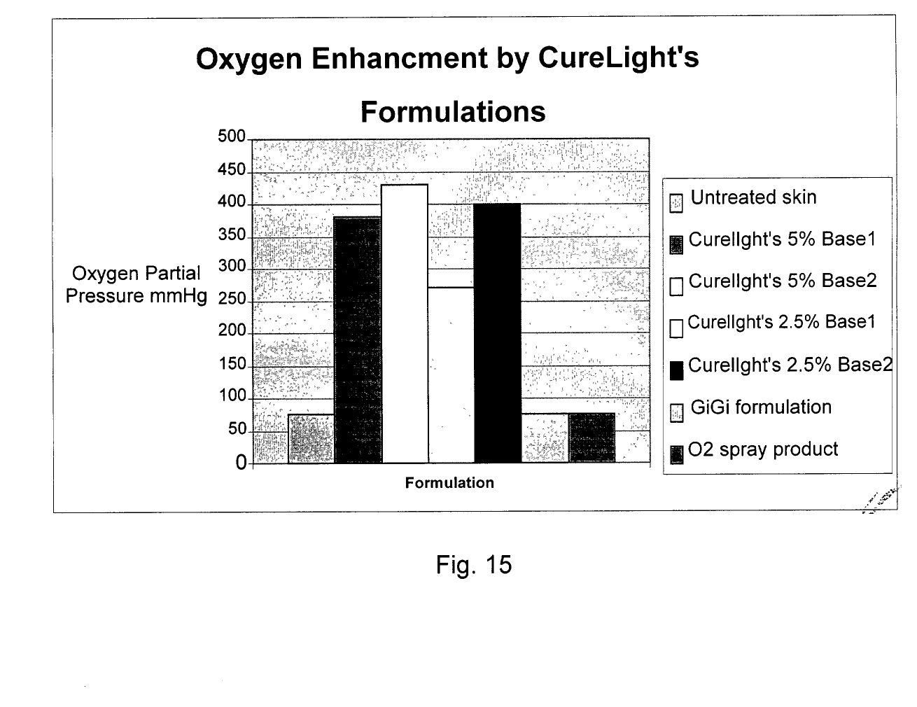

Tco₂M transcutaneous Co₂/o₂ Monitor Model 860 by Novametrix Medical Systems was used to measure cutaneous oxygen. Transcutaneous oxygen was measured with an oxygen sensor consisting of 2 parts; [1] A modified Clark type polarographic electrode, a silver anode and platinum cathode, electrolyte and a oxygen permeable membrane [2] a heating section with two precision thermistors for measuring and controlling the sensor temperature. Results, shown in FIG. 15, are as follows.

| | |
|---|---|
| Control measurement (nothing applied) | 77 mmHg |
| Invented formulation 5% base I (prepared May 1999) | 380 mmHg |
| Invented formulation 5% base II (prepared May 1999) | 430 mmHg |
| Invented formulation 2.5% base I (prepared Jan. 1999) | 270 mmHg |
| Invented formulation .5% base I (prepared Jan. 1999) | 400 mmHg |
| Other “Oxygen enhancing” commercial formulation | 75 mmHg |
| O2 Spray Product | 75 mmHg |

Formulations are steady for at least 9 months.

In Vitro Testing on Reduction of Pro Inflammatory Cytokines

Inflammatory processes in the human body are regulated by a complex set of soluble molecules that are called cytokines. Some of the cytokines such as INF-gamma and TNF-alpha cause cutaneous and other human cells to produce and release molecules that directly cause inflammation. One of these PRO-inflammatory molecules is Interleukine-1 alpha (IL-1 alpha).

It is known the Ultraviolet light is able to reduce induction of PRO inflammatory Cytokines. In another lab study we tried to find out if high intensity UV free blue visible light has also a direct anti inflammatory effect on human skin-un-related to its ability to photodestruct acne bacteria.

We used two different cell lines: HaCaT (produced by spontaneous immortalization of genetically altered cell line) and hTERT (obtained by stable transfection of primary cell culture with hTERT resulting in expression of telomerase catalytic subunit and immortalization). Cell cultures were treated with INF-gamma and TNF-alpha and exposed to UVB light (310 nm at 50 mJ/cm2, which is comparable to the dose used in treatment of some inflammatory skin conditions, such as psoriasis) and UV free visible blue light (405-440 nm at 90 mW/cm2. The expression of IL-1 alpha and ICAM-1 was measured by quantitative ELISA at 48 hours. We found that exposure to high intensity blue visible light reduced the release of the above Pro-inflammatory cytokines by 50% and 60% respectively. This was similar to the effect caused by UV light in doses that are used in phototherapy of other skin diseases such as Atopic Dermatitis and Psoriasis (Reduced Pro-inflammatory cytokines by 70% and 80% respectively).

This in vitro study proves that high intensity blue visible light has a very significant direct anti inflammatory effect on human cells. Its clinically proven high efficacy in Acne phototherapy is most probably based on both its photo destructive effect on acne bacteria thorough its porphyrines and on its direct anti inflammatory effect on human keratinocytes. We use this in vitro discovery to achieve a rapid decrease of post acne inflammatory condition which still exists after the eradication of acne bacteria. This inflammatory condition was in prior art was addressed only after the eradication of p. acnes bacteria with the aid of topical or intralesional corticosteroids, high intensity blue visible light was not used for that purpose.

In addition to its anti inflammatory use on the acne sites the described cytokine reducing UV free narrow band high intensity blue visible light apparatus may serve as an efficient phototherapeutic tool for a spectra of inflammatory conditions such as skin ulcers, cutaneous autoimmune diseases such as Lupus Erythematosus, soft tissue and gums inflammation and Musculoskeletal and neuralgic traumatic and inflammatory conditions.

It is to be understood that the invention is not limited in its applications to the details of construction or drawings. The invention is capable of other embodiments, or of being practiced or carried out in various ways. Also, it is to be understood that the phraseology and terminology employed above is for the purpose of description and should not be regarded as limiting. While the invention has been described with respect to a limited number of embodiments, it will be appreciated that many variations, modifications and other applications of the invention may be made.

While the invention has been described with respect to a limited number of embodiments, it will be appreciated that many variations, modifications and other applications of the invention may be made.

What is claimed is:

1. An apparatus for treatment of a skin disorder, the apparatus comprising:
(a) at least one light source with spectral emittance concentrated in at least one specific narrow spectral band, wherein one spectral band is in the range of 405 to 440 nm;
(b) an optical system for collecting and shaping light emitted from said at least one light source; and
(c) an electronic unit to control parameters associated with said spectral emittance from said at least one light source.

2. The apparatus of claim 1, wherein said parameters include at least one of a group including duration, power and emitted spectral bands of said light source emittance.

3. The apparatus of claim 1, further comprising:
a mechanical fixture for holding said light source at an adjustable distance and direction related to a treatment area.

4. The apparatus of claim 1, wherein an illumination energy of said light source flux, is higher than a predetermined threshold level.

5. The apparatus of claim 1, wherein the illumination of said light source flux, In the entire UV spectrum is less than a predetermined UV safety energy threshold level.

6. The apparatus of claim 5, wherein the illumination flux of said light source in the UV spectrum is less than 0.1 microwatt/cm² in the spectral range of 200-400 nm.

7. The apparatus as in claim 4, wherein said threshold level is a level required for biological destruction of acne and seborrhea causing factors.

8. The apparatus as in claim 4, wherein said threshold level is a level required for biological destruction of p. acne bacteria at a rate faster than their rate of proliferation.

9. The apparatus of claim 4, wherein said illumination energy threshold level of said illumination light source is at least 40 mw/cm² at a distance from the light source of 30 cm.

10. The apparatus of claim 4, wherein said illumination energy threshold level of said illumination light source is at least 40 mw/cm² at a distance from the light source of 30 cm.

11. The apparatus of claim 4, wherein said illumination energy threshold level is a level facilitated by a high energy output of a non coherent illumination source; and wherein said illumination source is any combination of radiation sources selected from the group including at least a gas discharge metal halide lamp, an excimer lamp, a LED diode matrix and a diode laser matrix.

12. The apparatus of claim 1, wherein the illuminated area on a patient body by said light source comprises an illumination area large enough to illuminate an infected typical size skin area from a fixed position of said light source related to said skin area.

13. The apparatus of claim 7, wherein the illuminated area on a patient body by said light source is characterized by an energy flux that is above said threshold level and that is large enough in its dimensions to cover and treat in a single exposure session an entire human body part and an entire infected skin area from at least a single fixed position of said light source related to said organ and skin area.

14. The apparatus of claim 12, wherein said illuminated area is at least 400 cm².

15. The apparatus of claim 1, further comprising an illumination head comprising at least two converging collimated beams from at least two directions, each of said beams generated by a separate light source positioned at a distance from said other at least one light source.

16. The apparatus of claim 1, further comprising:
a computer controlled imaging unit for imaging an illuminated treated area and for monitoring by counting lesions on said treated area, using computerized counting techniques.

17. The apparatus of claim 16, further comprising:
a computer controlled display unit for displaying said imaged illumination treated area, wherein counting is carried out by an operator marking lesions on the display of said illumination treated imaged surface area.

18. The apparatus of claim 16, further comprising:
a computer controlled display unit for displaying said imaged illumination treated area and for enabling computer lesions counting by image processing techniques to detect and count each lesion in the illumination treated imaged surface area.

19. The apparatus of claim 16, wherein the score of said computer lesion counting is recorded in a computer memory to enable monitoring the lesion healing process through a series of consecutive treatments.

20. The apparatus of claim 16, wherein said computer controlled imaging unit display image includes at least one of a group including a graph of the number of counted lesions versus accumulated treatment time and a table consisting of number of counted lesions in each treatment session.

21. The apparatus of claim 1, further comprising:
at least one optical element of a group comprising a liquid filled light guide, a solid transparent light guide, a fiber bundle light guide and an array of lenses and mirrors for collecting and conducting the said light source radiation and illuminating the skin treated area at an adjustable distance, energy density and direction.

22. The apparatus of claim 1, wherein said at least one light source is a Gallium, Mercury and halides gas mixture discharge lamp with peak emission in the 405-440 spectral band.

23. The apparatus of claim 1, wherein said at least one light source is an Excimer lamp with peak emission in the 405-440 spectral band.

24. The apparatus of claim 1, wherein said at least one light source is selected from the group including a Gallium, Mercury and halides gas mixture discharge lamp with peak emission in the 405-440 spectral band, an Excimer lamp with peak emission in the 405-440 spectral band, an Ion Krypton gas laser with a spectral emission in the range 405 to 440 nm, and a diode, wherein said diode is selected from the group consisting of violet/blue laser diodes, and light emitting diodes (LED) with narrow spectral band emission in the range 405-440 nm, or any combination thereof.

25. The apparatus of claim 1, wherein the light of said at least one light source is collected and further projected by at least one reflector, wherein said reflector is selected from the group comprising of an elliptical cross-section cylindrical reflector, parabolic cross-section cylindrical reflector, and an asymmetric aspheric reflector.

26. The apparatus of claim 1, wherein the light of said at least one light source is collected and further collimated by a set of two orthogonal cylindrical lenses.

27. The apparatus of claim 1, wherein the light of said at least one light source is collected by an elliptical cross-section reflector having a first focal point and a second focal point and wherein said light source is disposed at said first focal point and has disposed at said second focal point a slit shape aperture of a slit to circular beam shaping and conducting light guide.

28. A method of treating a skin disorder, comprising:
providing a light radiation source having spectral characteristics of at least one of a group of narrow spectral bands consisting of violet/blue (400-450 nm), red (630-670 nm) and green (520-550 nm) light said light source having energy output flux over a predetermined threshold level;
illuminating said skin area with said light radiation source; and
additionally illuminating said skin area after a predetermined time period.

29. The method of claim 28, wherein said threshold level is above 40 mw/cm² from a treatment distance of 30 cm or 50 mw/cm² from a distance of 25 cm.

30. A method of treating a skin disorder, comprising:
providing a light radiation source having spectral characteristics of at least one of a group of narrow spectral bands consisting of violet/blue (405-440 nm), red (630-670 nm) and green (520-550 nm) light;
applying a compound to a skin area;
illuminating said skin area with said light radiation source; and
additionally illuminating said skin area after a predetermined time period.

31. A method according to claim 30, wherein said compound is selected from a group consisting of a topical oxygen transporting perfluoroocarbon, an oxidative agent, a keratolytic agent and a Methylene blue solution.

32. A method according to claim 28, wherein said predetermined time period is at least 24 hours.

33. A method according to claim 28, wherein said skin disorder is one of a group including acne and seborrhea.

34. A method according to claim 28, further comprising:
pretreatment application of at least one compound selected from said compound group;
concentrating said light on said skin area by an optical system and a mechanical fixture; and
exposing said skin area at specific time intervals.

35. A method according to claim 28, wherein said time interval is 1-5 weekly exposures to violet/blue light for typically 2-10 weeks, with a minimum 24 hour's time gap between exposures.

36. A method according to claim 28, wherein said step of illuminating is accomplished by projecting on said skin area with an illumination power in the range of 10 mW/cm² to 500 mW/cm² of violet/blue light radiation.

37. The method according to claim 28, wherein said compound is hydrogen peroxide in the concentration of 1-10% by weight and the concentration of salicylic acid is 1-10% by weight.

38. The method according to claim 34, wherein said pretreating is carried out daily.

39. The method according to claim 34, wherein said pretreating is carried out immediately before light exposure.

40. The method according to claim 34, wherein the material selected from the group consisting of oxidative and keratolytic compounds is in an aqueous gel.

41. The method according to claim 30, wherein the material selected from the group consisting of oxidative and keratolytic compounds is in oil in water emulsion.

42. The method according to claim 30, wherein the oxidative and/or keratolytic compound is within a material selected from the group consisting of a liposome and a positively charged submicron emulsion.

43. The method according to claim 30, wherein the oxidative and/or keratolytic compounds is in a Propylene glycol 10-50% base.

44. The method according to claim 30, wherein the oxidative compound is an oil in water emulsion mixed with molecular oxygen that is sprayed continuously on the skin before or during light exposure.

45. The method according to claim 30, wherein Methylene blue 0.1-5% in distilled water or gel bases is applied to the skin before or during light exposure.

46. The apparatus of claim 1, wherein said treatment of a skin disorder is for the reduction of inflammation, and wherein said one spectral band is further enhanced to the 400-450 nm spectral range.

47. The apparatus according to claim 4, wherein said treatment of a skin disorder is for the reduction of inflammation.

48. The method according to claim 28, wherein said skin disorder is inflammation, said light radiation source having spectral characteristics that contain at least the narrow band of violet/blue (400-450 nm); and said light source having energy output flux over a predetermined threshold level.

49. The method according to claim 48, wherein said power flux predetermined threshold level is higher than 10 mw/cm².

50. The method according to claim 49, wherein treated area is larger than 2 cm×2 cm.

51. The method according to claim 48, wherein said light radiation source is capable of reducing the level of extra cellular pro-inflammatory cytokines.

52. The method according to claim 51, wherein said cytokines are reduced by an amount of at least 70% of the reduction achieved by UVB at the same energy radiation conditions.

53. The method according to claim 24, wherein said light radiation source is capable of treating skin ulcers.

54. The apparatus according to claim 46, wherein said source is capable of treating any skin disorder from the group including soft tissue inflammation, Muscular inflammation, Post traumatic soft tissue and muscular pains, arthralgia, skin ulcers such as diabetic ulcers and stasis ulcers, contact dermatitis, atopic dermatitis and systemic and localized scleroderma.

55. The method according to claim 48, wherein said treatment is performed in conjunction with a topical formulation compound which enhances lesional tissue partial oxygen pressure.

56. The method according to claim 48, wherein said treatment is performed in conjunction with Hyperbaric oxygen therapy.

57. The method according to claim 48, wherein said treatment is performed in conjunction with mechanical debridment of tissue i.e. mechanical, chemical, by high pressure water jet or ultrasonic waves

58. The method according to claim 55, wherein said compound is selected from a group including a topical oxygen transporting perfluoroocarbon, an oxidative agent including Hydrogen Peroxide formulation, a keratolytic agent and a Methylene blue solution.

59. The method according to claim 48, wherein said predetermined time period is 1-5 weekly exposures to violet/blue light for typically 1-10 weeks, with a minimum 24 hour's time gap between exposures.

60. The method according to claim 48, wherein said step of illuminating is accomplished by projecting on said skin area with an illumination power in the range of 10 mW/cm² to 100 mW/cm² of violet/blue light radiation.

61. The apparatus according to claim 46, wherein said apparatus radiation spectrum is UV free.

62. The apparatus according to claim 46, wherein said at least one light source is a Gallium, Mercury and halides gas mixture discharge lamp with peak emission in the 400-450 spectral band.

63. The apparatus according to claim 46, wherein said light source illumination flux in the 400-450 nm wavelength range is at least 10/mw/cm².

64. The apparatus of claim 1, wherein said at least one light source is selected from the group including Ion Krypton gas laser with a spectral emission in the range 400 to 450 nm, and a diode, wherein said diode is selected from the group consisting of violet/blue laser diodes, and light emitting diodes (LED) with narrow spectral band emission in the range 400-450 nm.

65. The apparatus according to claim 4, wherein said treatment of a skin disorder is for the treatment of acne, wherein said threshold level is selected as to enable the average reduction of at least 60% of the original number of acne lesions within 4 weeks; and without radiating any UV illumination on the treated skin area and without causing any damage to this skin.

66. The method according to claim 28, wherein said treatment of a skin disorder is for the treatment of acne, wherein said threshold level is selected as to enable the average reduction of at least 60% of the original number of acne lesions within 4 weeks; and without radiating any UV illumination on the treated skin area and without causing any damage to this skin.
